(19) United States
(12) Patent Application Publication
(10) Pub. No.: US 2015/0314103 A1
(43) Pub. Date: Nov. 5, 2015

(54) READY TO USE MEDICAL DEVICE WITH INSTANT ANTIMICROBIAL EFFECT

(71) Applicant: HOLLISTER INCORPORATED, Libertyville, IL (US)

(72) Inventors: David Hannon, Ballina (IE); Thomas H. Gilman, Spring Grove, IL (US)

(73) Assignee: HOLLISTER INCORPORATED, Libertyville, IL (US)

(21) Appl. No.: 14/650,156

(22) PCT Filed: Dec. 3, 2013
(86) PCT No.: PCT/US2013/072823

§ 371 Date: Jun. 5, 2015

Related U.S. Application Data
(60) Provisional application No. 61/735,127, filed on Dec. 10, 2012.

(51) Int. Cl. A61M 25/00; B65B 5/04; B65B 7/02; A61B 19/02

(52) U.S. Cl. CPC A61M 25/002; A61M 25/0045; A61B 19/026; A61M 25/0009; B65B 5/04; B65B 7/02; A61B 2019/0274; A61B 2019/0267

(57) ABSTRACT

A catheter or other medical device is provided in a package having a source of liquid capable of changing phase into a vapor that can activate a coating and/or be introduced into a matrix of the material that forms the catheter to thereby activate or release an antimicrobial component within the matrix so it can begin acting instantly after the package is opened and the catheter or other medical device is placed into use.

The present application claims priority to and the benefit of U.S. Provisional Application No. 61/735,127, filed Dec. 10, 2012, which is hereby incorporated herein by reference.

### FIELD OF THE DISCLOSURE

The present disclosure relates generally to ready to use medical devices that have fast acting or instant antimicrobial properties right out of the package and upon insertion into the human body, and more particularly, to vapor hydrated medical devices, such as ready to use vapor hydrated urinary catheters suitable for use in the drainage of urine from the bladder.

### BACKGROUND OF THE DISCLOSURE

Medical professionals are very aware that infections secondary to the use of indwelling catheters represent a significant healthcare burden. Clinical research indicates that with current indwelling catheters a significant percentage of these infections are due to the entry of bacteria into the urinary tract as the catheter is inserted and advanced through the urethra. Once bacteria have entered the urethra, they can easily move up the urethra and into the bladder causing the patient to develop a urinary tract infection.

It is also well known that after an indwelling catheter has been inserted and is in place that bacteria can enter and move up the lumen of the indwelling catheter. Thus, the lumen of the catheter may provide another pathway for bacteria to reach the bladder of a patient and cause a urinary tract infection.

Moreover, highly trained, highly focused nursing care and sterile conditions are not available in all patient care settings. For this reason, it is known to be quite common for the outer surface and lumen of an indwelling catheter to become contaminated with bacteria during handling and insertion. With such contamination, it is likely the bacteria will reach the bladder and the patient will develop a urinary tract infection as a consequence.

Contamination of medical devices is a common problem. One method of combating such contamination has been to incorporate an antimicrobial agent into the matrix of the material of the medical device. There is, however, an issue with the diffusion rate of active components from the matrix of materials in many medical devices. The active component near the surface of a medical device typically makes contact with a liquid and elutes from the surface but, since the active component is in a solid matrix rather than at the surface, the diffusion rate for the active component may be very slow. Thus, for many medical devices this can lead to a delayed antimicrobial effect as it takes some time for the active component to elute from the surface after initial contact with the liquid. The desired antimicrobial effect also may diminish, resulting in a reduction of antimicrobial effectiveness, as the elution rate slows.

In an effort to address at least some of these problems, there have been proposed various different techniques such as applying an outer layer or coating of a hydrogel containing an antimicrobial component to an indwelling catheter to provide infection control and incorporating anti-infective or antimicrobial agents into the matrix of polymers used to form indwelling catheters but none of them have been entirely effective in addressing the various problems discussed above.

Generally, in either case, the anti-infective or antimicrobial component or agent will not diffuse to the surface of the coating or polymer materials so long as the polymer is in a state or condition of drawing liquid into the matrix to hydrate the polymer. Generally, the anti-infective or antimicrobial component or feature will diffuse to the surface only after the coating or the polymer matrix is hydrated for a period of time. Unfortunately, it takes time for this diffusion to occur and reach a point of equilibrium where the anti-infective or antimicrobial component or agent is substantially evenly dispersed.

Typically, however, the coating or the polymer matrix undergoes hydration only immediately before the use of an indwelling catheter. Thus, there will be insufficient time for there to be sufficient diffusion of the anti-infective or antimicrobial component or agent to reach equilibrium. As a result, immediately after hydration and insertion of the catheter, there will usually be an insufficient amount of the anti-infective or antimicrobial component or agent at the surface to be effective.

In line with the foregoing, there has been a problem not only in connection with indwelling catheters but also with many other medical devices, to provide an instant antimicrobial effect inside and/or outside the lumen of a catheter and on any critical surface of other medical devices, so the catheter or other medical devices are ready to use by medical professionals without the need to first prepare them for use.

### SUMMARY OF THE DISCLOSURE

The present disclosure is directed to a catheter or other medical device which is in ready to use condition and incorporates an antimicrobial component into the material of the catheter or device and/or into a coating of the catheter or other medical device.

For this purpose, the catheter or other medical device is provided in a package having a source of vapor donating liquid medium that is capable of changing phase into a vapor that can activate the material and/or be introduced into the matrix of the material to thereby activate or release the antimicrobial component(s) so it can begin acting instantly after the package is opened and the indwelling catheter or other medical device is placed into use.

### BRIEF DESCRIPTION OF THE DRAWINGS

FIG. 1 is a schematic plan view of a package containing a ready to use catheter or other medical device having instant antimicrobial effect;

FIG. 2 is a cross-sectional view taken generally along the line 1-1 of FIG. 1.

FIG. 3 is a top plan view, partially broken away, of another embodiment of a package containing a ready to use catheter or other medical device having an instant antimicrobial effect; and

FIG. 4, is a top plan view, partially broken away, of another embodiment of a package containing a ready to use catheter or other medical device having an instant antimicrobial effect.

### DETAILED DESCRIPTION OF THE PRESENT DISCLOSURE

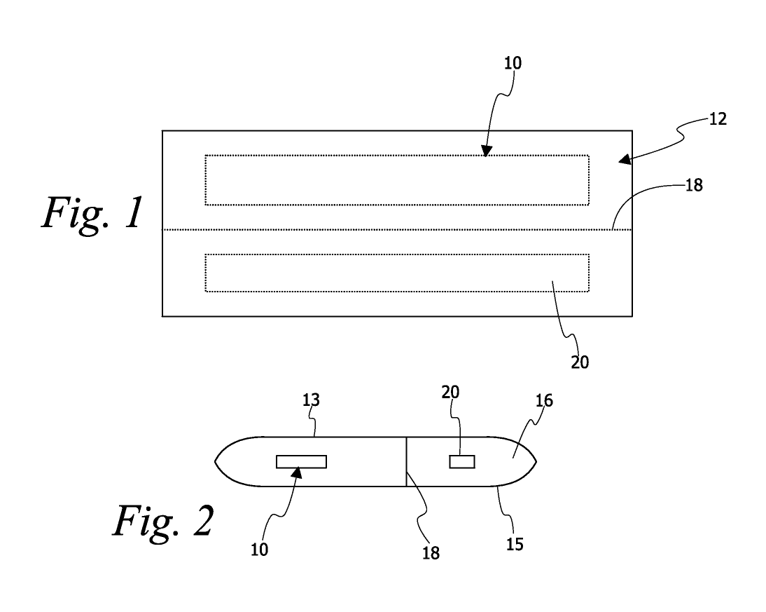

In FIG. 1, a medical device 10, such as an intermittent or indwelling catheter, having instant antimicrobial effect upon opening of the package is contained within a package 12. The package 12 may be formed from one or more liquid and gas impermeable materials such as an aluminum foil, aluminum foil laminate or a polymer(s). The package 12 may be sealed to form a liquid and gas tight package. In one exemplary embodiment, the package 12 may include two cavities 14 and 16 (see FIG. 2). In the embodiment shown, the package 12 may include a liquid impermeable and gas permeable membrane 18 which serves to divide the interior of the package 12 into the two cavities 14 and 16.

Referring to FIGS. 1 and 2, it will be seen that the ready to use medical device 10 is disposed within the cavity 14. It will also be seen that the cavity 16 may, optionally, include a fabric or open-cell polymeric foam liquid sequestering element 20. When present, the liquid sequestering element 20 may either be integrally associated with one of the walls 13 and 15 or may be loose within the cavity 16.

The cavity 16 and liquid sequestering element 20, when present, may be sized to contain an amount of liquid water capable of changing phase into a sufficient amount of water vapor to form and maintain a 100% relative humidity atmosphere within the package 12. Specifically, the cavity 16 and liquid sequestering element 20, when present, may be sized to be capable of receiving and holding a sufficient amount of liquid water to maintain a 100% relative humidity atmosphere within both of the cavities 14 and 16 for a desired product shelf life. In this connection, the water vapor which is produced within the cavity 16 after the package 12 has been assembled and sealed is capable of passing through the gas permeable membrane 18 into the cavity 14 to cause this to occur. The water vapor in cavity 14 vapor hydrates the medical device 10. The distribution of the package may be delayed for a time sufficient to fully hydrate the medical device and/or to allow the antimicrobial agent to sufficiently diffuse to the surface of the matrix material.

As mentioned above, the sequestering element is optional and in one embodiment the liquid sequestering element 20 may be eliminated and cavity 16 may contain a vapor donating liquid medium without the need for sequestering element 20.

The cavity 16 may be integrated with the package wherein a wall of the package forms part of the cavity. In other embodiments, cavity 16 may be formed separately from the package material.

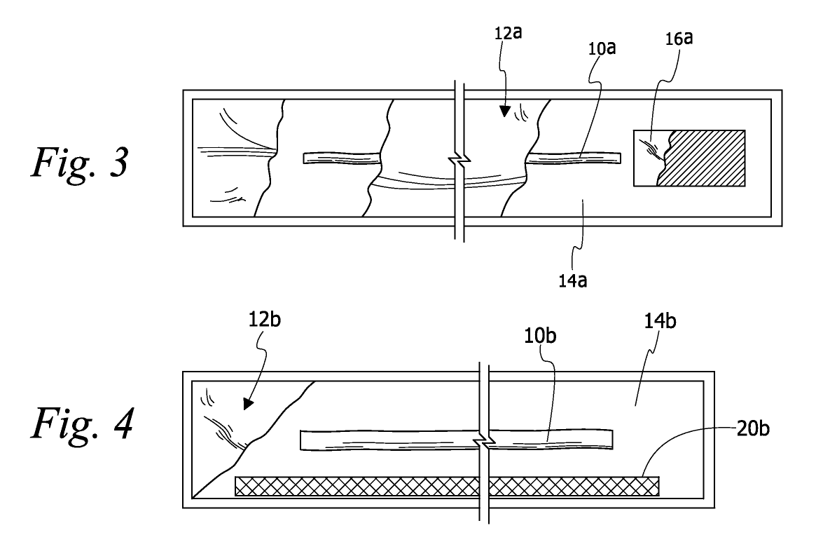

FIG. 3 illustrates another embodiment of a package 12a wherein a medical device 10a, such as a catheter, is located within a liquid and gas tight cavity 14a. In this embodiment, the cavity 16a containing the amount of vapor donating liquid of package 12a may include one or more liquid filled sachets placed within the cavity 14a of package 12a. The sachets may be at least partially made of a liquid impermeable, vapor permeable material which allows vapor produced from some of the amount of the vapor donating liquid to pass through the material and into cavity 14a, thereby hydrating the medical device 10a. The cavity 16a, such as the illustrated sachet, may optionally also include a liquid sequestering element.

Turning to FIG. 4, in another embodiment, the membrane may be eliminated so that the package 12b defines a liquid and gas tight cavity 14b that includes both the medical device 10b and liquid sequestering element 20b wherein a vapor donating medium may be sequestered in liquid form in liquid sequestering element 20. Some of the sequestered amount of liquid changes phase into a vapor in cavity 14b wherein the vapor hydrates the medical device.

While shown schematically in Figures, it will be appreciated that the medical device 10, 10a and 10b may take various forms and may have inner and/or outer surfaces upon which there are coatings having an antimicrobial component, or the antimicrobial component can be incorporated into the matrix of the material from which the medical device 10 is formed.

More specifically, and especially in the case of intermittent and indwelling catheters, a hydrophilic coating containing an oligodynamic component may be provided on the outer surface of the catheter. The oligodynamic component may comprise ions such as silver in the lubricious coating which have an antimicrobial effect so when the hydrophilic coating on the catheter is hydrated in a vapor atmosphere before distribution to an end user there is diffusion of the silver ions so they are not only present on the catheter surface, but they reach a state of equilibrium in which they are substantially evenly dispersed throughout the hydrophilic coating on the catheter. As a result of this diffusion, the watery catheter surface has an immediate antimicrobial effect, and the catheter is instantly ready for use upon opening of the package as compared to a catheter that has not been pre-vapor hydrated within the package.

As a particular advantage, the use of oligodynamic ions is widely accepted for medical device antimicrobial applications with one of the primary advantages being the lack of resistant bacteria. The device and methods of the present disclosure advantageously results in the presence of the ions at the site to be protected, i.e., the urethra following insertion of an indwelling catheter, and the ions being present and evenly dispersed throughout the hydrophilic coating and on the outer surface of the catheter. Having the ions present at the site to be protected and evenly dispersed throughout the hydrophilic coating and outer surface of the catheter achieves both an instant antimicrobial effect after the package is opened and during the insertion of the catheter and a continuing antimicrobial effect while the catheter remains indwelling in the urethra during use. This is achieved by equilibrium being reached between the concentration of silver ions present on the watery catheter outer surface and the silver ions present in the lubricious hydrophilic coating.

As a result of this equilibrium, the silver content within the hydrophilic coating will not be depleted to any significant degree and, thus, will not adversely affect the continuing antimicrobial effect during the duration the catheter remains indwelling because the amount of water on the watery catheter surface is small.

As will be appreciated, the presence of the antimicrobial effective ingredient on the watery catheter surface provides an instant antimicrobial effect as the ingredient will be in contact with the urethra both during and immediately after insertion of the catheter. While the presence of the antimicrobial effective ingredient dispersed within the hydrophilic coating will make the antimicrobial effective ingredient available for the entire duration the catheter remains indwelling which can typically run up to 30 days in the case of a Foley catheter.

In addition to incorporating an oligodynamic component into a hydrophilic coating on the surface of a catheter or other medical device, the present disclosure also contemplates the active component being contained within the material of the catheter or device. When the active component is contained in the material, the catheter or device may or may not have a lubricious hydrophilic coating on a surface. However, the active component will initially be contained within the polymer matrix of the material for the catheter or device, and the water vapor produced by a change of phase of the liquid water in the package will diffuse or assist in moving the active component into and through the hydrophilic coating and to the surface of the hydrophilic coating, when a hydrophilic coating is present.

In the case of intermittent and indwelling catheters, the catheter will be ready to use because diffusion of the active component will have taken place into the watery layer on the outer surface of the catheter before it reaches the end user. The watery layer on the catheter outer surface will therefore have a quicker antimicrobial effect than a catheter hydrated immediately prior to insertion. However, it will also be the case that there will have been diffusion of the active component onto the inner surface of the catheter, which defines the catheter's inner lumen, due to its presence in the polymer matrix of the material of the catheter. This provides an antimicrobial effect within the inner lumen which reduces the risk of bacteria moving through the inner lumen and into the bladder.

From the foregoing, it will be seen that the present disclosure therefore also addresses the problem of having an active ingredient on the inner surface defined by the lumen of an indwelling catheter. This is achieved by having the active ingredient contained within the polymer matrix of the catheter material. As an additional benefit, the water molecules which are present in the water vapor help to penetrate into the polymer matrix to cause the active ingredient to avoid any lull in elution.

More specifically, there is an issue with the diffusion rate of the active component from the polymer matrix of the material for many medical devices. The active component near the surface makes contact with liquid when a medical device is being prepared for use and elutes from the surface but, since the active component below the surface is in a solid matrix, the diffusion rate becomes very slow which can lead to a fall off in the elution rate. When a fall off in the elution rate is experienced, there is less available active component and a consequent reduction in overall effectiveness.

Because the water molecules will penetrate into the polymer matrix as a result of the presence of the water vapor in the package, and thereby assist the diffusion of the active component contained in the polymer matrix to the surface of the indwelling catheter or other medical device, the present disclosure is thereby capable of ensuring against any lull in elution.

In order to better understand the details of vapor hydration of a hydrophilic coating on a catheter, it is instructive to make reference to commonly owned U.S. Pat. No. 7,380,658. The catheters disclosed in that patent are single use hydrophilic coated intermittent urinary catheters whereas the present disclosure contemplates indwelling, or Foley, urinary catheters of the type which are most commonly used in medical facilities for long-term catheterization or, alternatively, other medical devices. In either case, U.S. Pat. No. 7,380,658 is incorporated by reference in its entirety for teaching production of a 100% relative humidity atmosphere within a medical device product package.

The teachings of U.S. Pat. No. 7,380,658 are therefore relevant to all aspects of the present disclosure since all that is required for either the vapor hydration of a hydrophilic coating containing an antimicrobial component on a ready to use indwelling catheter or other medical device or causing water molecules to penetrate into the polymer matrix of a material for a ready to use indwelling catheter or other medical device is to have a 100% relative humidity atmosphere within the package for the catheter or device.

In a first aspect, the present disclosure relates to a ready to use medical device assembly comprises a package at least partially made of liquid and gas impermeable material; a medical device disposed within the package, the medical device at least partially formed from a matrix material having an antimicrobial agent incorporated therein; and a vapor donating liquid medium disposed within the package, wherein the vapor donated from the liquid medium hydrates the matrix material.

In a second aspect, the assembly of aspect 1 wherein the package forms a sealed package and the medical device is disposed within the cavity.

In a third aspect, the assembly of any one of the preceding aspects wherein the matrix material comprises a polymer.

In a fourth aspect, the assembly of any one of the preceding aspects wherein the matrix material comprises a hydrophilic polymer.

In a fifth aspect, the assembly of any one of the preceding aspects wherein the antimicrobial agent comprises an oligodynamic agent.

In a sixth aspect, the assembly of any one of the preceding aspects wherein the antimicrobial agent comprises silver.

In a seventh aspect, the assembly of any one of the preceding aspects wherein the vapor donating liquid medium is substantially separated from the catheter.

In an eighth aspect, the assembly of any one of the preceding aspects wherein the vapor donating liquid medium is sequestered by a liquid sequestering element.

In a ninth aspect, the assembly of any one of the preceding aspects wherein the package includes a vapor permeable, liquid impermeable membrane separating the vapor donating liquid medium from the catheter.

In a tenth aspect, the assembly of aspect 9 wherein the vapor permeable, liquid impermeable membrane at least partially defines a cavity containing the vapor donating liquid medium.

In an eleventh aspect, the assembly aspect 9 wherein the vapor permeable, liquid impermeable membrane at least partially forms one or more sachets disposed within the package.

In a twelfth aspect, the assembly of any one of the preceding aspects wherein the medical device is a catheter.

In a thirteenth aspect, the assembly of aspect 12 wherein the catheter is an indwelling catheter.

In a fourteenth aspect, the assembly of any one of the preceding aspects wherein the matrix material of the medical device containing the antimicrobial agent is an exterior hydrophilic layer or coating on the medical device.

In a fifteenth aspect, a method of manufacturing a ready to use catheter comprises placing a catheter into a package made from a gas and liquid impermeable material, wherein the catheter is made from a matrix material having an antimicrobial agent incorporated therein; placing a vapor donating liquid medium within the package; sealing the package; hydrating the matrix material with vapor donated from the donating medium so that the antimicrobial agent diffuses to the surface of the matrix material.

In a sixteenth aspect, the method of aspect 15 wherein the package is sealed to form a sealed cavity containing the medical device.

In a seventeenth aspect, the method of any one of aspects 15 and 16 wherein the matrix material comprises a polymer.

In an eighteenth aspect, the method of any one of aspects 15-17 wherein the matrix material comprises a hydrophilic polymer.

In a nineteenth aspect, the method of any one of aspects 15-18 wherein the antimicrobial agent comprises an oligodynamic agent.

In a twentieth aspect, the method of any one of aspects 15-19 wherein the antimicrobial agent comprises silver.

In a twenty-first aspect, the method of any one of aspects 15-20 wherein the vapor donating liquid medium is substantially separated from the catheter.

In a twenty-second aspect, the method of any one of aspects 15-21 wherein the vapor donating liquid medium is sequestered by a liquid sequestering element.

In a twenty-third aspect, the method of any one of aspects 15-22 wherein the package includes a vapor permeable, liquid impermeable membrane separating the vapor donating liquid medium from the catheter.

In a twenty-fourth aspect, the method of aspect 23 wherein the vapor permeable, liquid impermeable membrane at least partially defines a cavity containing the vapor donating liquid medium.

In a twenty-fifth aspect, the method of aspect 23 wherein the vapor permeable, liquid impermeable membrane at least partially forms one or more sachets disposed within the package.

In a twenty-sixth aspect, the method of any one of aspects 15-25 wherein the medical device is a catheter.

In a twenty-seventh aspect, the method of aspect 26 wherein the catheter is an indwelling catheter.

In a twenty-eighth aspect, the method of any one of aspects 15-27 wherein the matrix material of the medical device containing the antimicrobial agent is an exterior hydrophilic layer or coating on the medical device.

In a twenty-ninth aspect, the method of any one of aspects 15-28 further including delaying distribution of the package after sealing the package for a period of time sufficient to completely vapor hydrate the medical device and to allow the antimicrobial agent to sufficiently diffuse to the surface of the matrix material.

1. A ready to use medical device assembly comprising:
a package at least partially made of liquid and gas impermeable material;
a medical device disposed within the package, the medical device at least partially formed from a matrix material having an antimicrobial agent incorporated therein; and
a vapor donating liquid medium disposed within the package, wherein the vapor donated from the liquid medium hydrates the matrix material.

2. The assembly of claim 1 wherein the package forms a sealed package and the medical device is disposed within the cavity.

3. The assembly of claim 1 wherein the matrix material comprises a polymer.

4. The assembly of claim 1 wherein the matrix material comprises a hydrophilic polymer.

5. The assembly of claim 1 wherein the antimicrobial agent comprises an oligodynamic agent.

6. The assembly of claim 1 wherein the antimicrobial agent comprises silver.

7. The assembly of claim 1 wherein the vapor donating liquid medium is substantially separated from the catheter.

8. The assembly of claim 1 wherein the vapor donating liquid medium is sequestered by a liquid sequestering element.

9. The assembly of claim 1 wherein the package includes a vapor permeable, liquid impermeable membrane separating the vapor donating liquid medium from the catheter.

10. The assembly of claim 9 wherein the vapor permeable, liquid impermeable membrane at least partially defines a cavity containing the vapor donating liquid medium.

11. The assembly claim 9 wherein the vapor permeable, liquid impermeable membrane at least partially forms one or more sachets disposed within the package.

12. The assembly of claim 1 wherein the medical device is a catheter.

13. The assembly of claim 12 wherein the catheter is an indwelling catheter.

14. The assembly of wherein the matrix material of the medical device containing the antimicrobial agent is an exterior hydrophilic layer or coating on the medical device.

15. A method of manufacturing a ready to use catheter, comprising:
placing a catheter into a package made from a gas and liquid impermeable material, wherein the catheter is made from a matrix material having an antimicrobial agent incorporated therein;
placing a vapor donating liquid medium within the package;
sealing the package; and
hydrating the matrix material with vapor donated from the donating medium so that the antimicrobial agent diffuses to the surface of the matrix material.

16. The method of claim 15 wherein the package is sealed to form a sealed cavity containing the medical device.

17. The method of claim 15 wherein the matrix material comprises a polymer.

18. The method of claim 15 wherein the matrix material comprises a hydrophilic polymer.

19. The method of claim 15 wherein the antimicrobial agent comprises an oligodynamic agent.

20. The method of claim 15 wherein the antimicrobial agent comprises silver.

21-25. (canceled)

26. The method of claim 15 wherein the medical device is a catheter.

27. (canceled)

28. (canceled)

29. The method of claim 15 further including delaying distribution of the package after sealing the package for a period of time sufficient to completely vapor hydrate the medical device and to allow the antimicrobial agent to sufficiently diffuse to the surface of the matrix material.
